(19) United States
(12) Patent Application Publication
(10) Pub. No.: US 2020/0181192 A1
(43) Pub. Date: Jun. 11, 2020

(54) DISULFIDE BIOCONJUGATION

(71) Applicant: The Regents of the University of California, Oakland, CA (US)

(72) Inventors: Ellen M. SLETTEN, Los Angeles, CA (US); Monica PENGSHUNG, Los Angeles, CA (US)

(21) Appl. No.: 16/608,587

(22) PCT Filed: Apr. 27, 2018
(86) PCT No.: PCT/US18/29871

§ 371 Date: Oct. 25, 2019

Related U.S. Application Data
(60) Provisional application No. 62/491,855, filed on Apr. 28, 2017.

(51) Int. Cl. C07K 1/107; C07K 1/13; C07C 317/14

(52) U.S. Cl. CPC C07K 1/1077; C07C 317/14; C07K 1/13

(57) ABSTRACT

Compounds and methods are provided for one-step functionalization of disulfide bonds in proteins.

### REFERENCE TO RELATED APPLICATIONS

This application claims the benefit of U.S. Provisional Application No. 62/491,855, filed Apr. 28, 2017, the contents of which are fully incorporated by reference herein.

### BACKGROUND

Protein modification is an expansive field and the question of how to conjugate compounds to biomolecules can be answered in many ways. One common residue for functionalization is cysteine, which are often paired in biomolecules as a disulfide bond. Current methodologies for modifying disulfide bonds require a two-step process that leaves the protein vulnerable to unfolding and ultimately loss of protein function. Though the field of cysteine modification has been widely explored, the field of disulfide modification of proteins has only recently begun. Brocchini and coworkers started the field with their use of bissulfones as a Michael acceptor for free cysteine residues (Brocchini et al., Nat. Chem. Biol. 2006, 2, 312). By providing a critical third step, bissulfones could re-bridge the broken disulfide bonds after the initial addition. Brocchini's work was followed by Baker and Caddick, who began advancing maleimides for disulfide re-bridging (Caddick S.; Baker J., et al., JACS 2010, 132, 1960. Baker et al., Scientific Reports 2013, 1525). By utilizing the fast kinetics of di-substituted maleimides, the reduction and addition step could be performed in quick succession, lowering the chances of protein unfolding and disulfide scrambling. However, these methods still pass through an initial reduction step before reaction with the maleimide linker, which has the potential to cause protein unfolding and loss of function. New methods for functionalizing disulfides are needed.

### SUMMARY OF THE INVENTION

The present disclosure provides compounds and methods for peptide modification through selective one-step conjugation of disulfide bonds.

In some aspects, the present disclosure provides compounds comprising a bicyclobutane moiety coupled to a payload or reactive moiety. In some embodiments, the bicyclobutane moiety is represented by formula I:

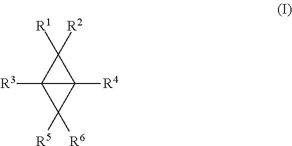

wherein:
R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, heteroarylsulfonyl, chromophore, fluorophore, or the payload or reactive moiety; and
further wherein any of R¹, R², R³, R⁴, R⁵, and R⁶, taken together with the carbon atoms that separate them, optionally complete one or more rings.

In some aspects, the present disclosure provides methods for preparing a conjugated biomolecule, comprising providing a biomolecule comprising a disulfide bridge; and reacting the disulfide bridge with a compound of any one of the preceding claims, thereby producing the conjugated biomolecule.

### BRIEF DESCRIPTION OF THE DRAWINGS

FIG. 1 shows a model example of a payload attached to a bicyclobutane molecule and its insertion into disulfide bonds present within antibodies through a radical mechanism.

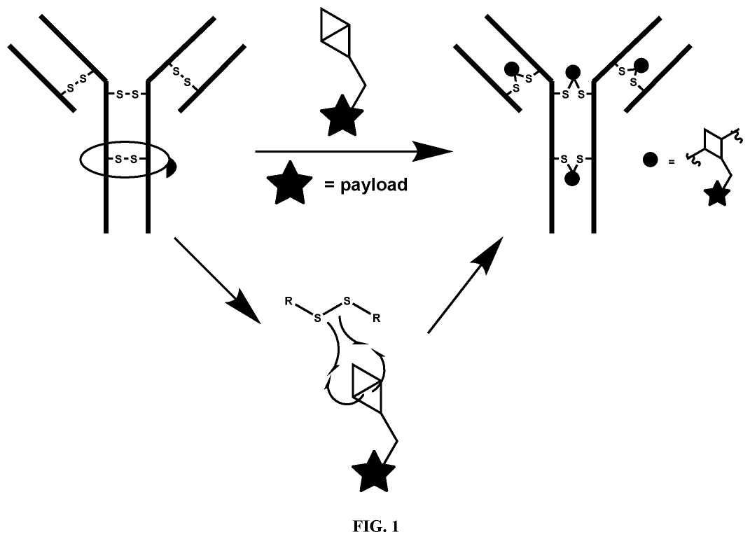

FIG. 2 shows a schematic of the insertion of the compounds of the present invention into the disulfide bonds of a polypeptide or protein.

### DETAILED DESCRIPTION OF THE INVENTION

In certain aspects, the present disclosure provides compounds comprising a bicyclobutane moiety coupled to a payload or reactive moiety. Such compounds provide a strained four-carbon system that is adapted to allow one-step, non-disruptive conjugation of disulfide bridges, for example in biomolecules. In certain embodiments, the bicyclobutane moiety is represented by formula I:

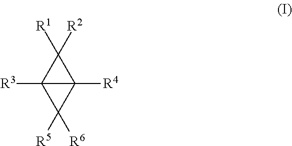

wherein:
R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, heteroarylsulfonyl, chromophore, fluorophore, or the payload or reactive moiety; and
   further wherein any of R¹, R², R³, R⁴, R⁵, and R⁶, taken together with the carbon atoms that separate them, optionally complete one or more rings.

In certain embodiments, of formula (I), R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, heteroarylsulfonyl, or the payload or reactive moiety. In certain embodiments, of formula (I), R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, or the payload or reactive moiety.

In certain embodiments of formula (I), any of R¹, R², R³, R⁴, R⁵, and R⁶, taken together with the carbon atoms that separate them, complete one or more rings.

In certain embodiments of formula (I), R¹, R², R³, R⁴, R⁵, and R⁶ are independently selected from H, alkyl, or the payload or reactive moiety; and any of R¹, R², R³, R⁴, R⁵ and R⁶, together with the carbon atoms that separate them, may be combined to complete one or more rings. In certain embodiments, at least one of R¹, R², R³, R⁴, R⁵ and R⁶ is arylsulfonyl or heteroarylsulfonyl, such as phenylsulfonyl. In certain embodiments, at least one of R², R³, R⁴, R⁵, and R⁶ is halo.

In certain embodiments, at least one of R¹, R², R³, R⁴, R⁵ and R⁶ is the payload or reactive moiety. In other embodiments, at least one of R¹, R², R³, R⁴, R⁵ and R⁶ is coupled to the payload or reactive moiety.

In certain preferred embodiments, R¹ is the payload or reactive moiety, and R³ is arylsulfonyl or heteroarylsulfonyl, such as phenylsulfonyl. In certain preferred embodiments of formula (I), R¹ is the payload or reactive moiety, and R³ is phenylsulfonyl. In other preferred embodiments, R¹ and R² are F, and R⁵ is the payload or reactive moiety.

In certain embodiments, the payload or reactive moiety coupled to the bicyclobutane moiety (for example, the bicyclobutane moiety of formula (I) as described above) is a payload moiety. In certain preferred embodiments, the payload moiety is a drug moiety, a detectable label, or a PEG moiety. In even more preferred embodiments, the detectable label is a fluorophore, a radiolabel, or an imaging agent.

In certain embodiments, the payload or reactive moiety coupled to the bicyclobutane moiety (for example, the bicyclobutane moiety of formula (I)) is a reactive moiety. In certain embodiments, the reactive moiety is a hydroxyl, haloacyl, an alkene, an alkyl halide, an alkyne, an amine, an aryl azide, an aryl halide, an azide, a carbodiimide, a carboxyl, a diene, a dienophile, a glyoxal, an imidoester, an isocyanide, a maleimide, an N-hydroxysuccinimidyl (NHS) ester, a phosphine, a tetrazine, or a thiol. In certain preferred embodiments, the reactive moiety is a haloacyl, an alkene, an alkyl halide, an alkyne, an amine, an aryl azide, an aryl halide, an azide, a carbodiimide, a carboxyl, a diene, a dienophile, a glyoxal, an imidoester, an isocyanide, a maleimide, an N-hydroxysuccinimidyl (NHS) ester, a phosphine, or a tetrazine.

In certain embodiments, the bicyclobutane moiety (for example, the bicyclobutane moiety of formula (I)) is a portion of a polycyclic structure selected from: (a) 2,4 methano-2,4-didehydroadamantane; (b) 1-(phenylsulfonyl)bicyclo[1.1.0]butane; (c) 2-methyl-bicyclo[1.1.0]butane; (d) tricyclo[4.1.0.0]heptane; or (e) 2,2-difluorobicyclo[1.1.0]butane. In certain preferred embodiments, the compound is:

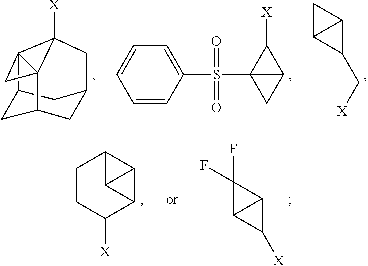

wherein X is the reactive or payload moiety.

In certain aspects, the present disclosure provides methods for preparing conjugated biomolecules, comprising providing a biomolecule comprising a disulfide bridge; and reacting the disulfide bridge with a compound of any one of the preceding claims, thereby producing the conjugated biomolecule. Advantageously, the present methods are capable of functionalizing biomolecules at disulfide bridges without proceeding through an intermediate step (such as a reduction) where the conformational constraint produced by the disulfide bridge is broken.

In certain embodiments, the reacting step is conducted in an aqueous solution, and optionally comprises adding a photoinitiator and irradiating the photoinitiator. In certain embodiments, the photoinitiator is DMPA or IRGACURE 2959:

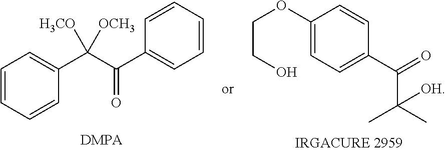 [SMILES: COC(OC)(C(=O)c1ccccc1)c1ccccc1 ; CC(C)(O)C(=O)c1ccc(OCCO)cc1]

In some embodiments, the bicyclobutane compound itself may be photosensitive or include a photosensitive moiety such that a separate photoinitiator will not be necessary. For instance, the bicyclobutane compound may be functionalized with a photosensitive arylsulfonyl or heteroarylsulfonyl group.

In certain aspects, the present disclosure provides a modified polypeptide comprising a moiety according to formula II:

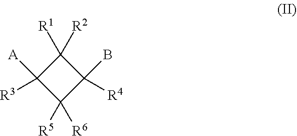

wherein:
A and B are sulfur atoms of cysteine residues of the polypeptide, wherein the sulfur atoms are capable of forming a disulfide bond in an unmodified form of the polypeptide;
R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, heteroarylsulfonyl, a chromophore, a fluorophore, or the payload or reactive moiety; and
further wherein any of R¹, R², R³, R⁴, R⁵, and R⁶, together with the carbon atoms that separate them, optionally complete one or more rings.

In certain embodiments, of formula (II), R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, heteroarylsulfonyl, or the payload or reactive moiety. In certain embodiments, of formula (II), R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, or the payload or reactive moiety.

In certain embodiments of formula (II), any of R¹, R², R³, R⁴, R⁵, and R⁶, together with the carbon atoms that separate them, complete one or more rings.

In certain embodiments of formula (II), A and B are cysteine residues in the same polypeptide. In other embodiments, A and B are cysteine residues in different polypeptides.

In certain embodiments, of formula (II), R¹, R², R³, R⁴, R⁵, and R⁶ are independently selected from H, alkyl, or the payload or reactive moiety; and any of R¹, R², R³, R⁴, R⁵, and R⁶, together with the carbon atoms that separate them, optionally complete one or more rings.

In certain preferred embodiments of formula (II), at least one of R¹, R², R³, R⁴, R⁵, and R⁶ is the payload or reactive moiety. In other preferred embodiments, at least one of R², R³, R⁴, R⁵ and R⁶ is coupled to the payload or reactive moiety. In certain embodiments, at least one of R², R³, R⁴, R⁵, and R⁶ is arylsulfonyl or heteroarylsulfonyl, such as phenylsulfonyl. In certain embodiments, at least one of R¹, R², R³, R⁴, R⁵, and R⁶ is halo.

In certain preferred embodiments, R¹ is the payload or reactive moiety, and R³ is arylsulfonyl or heteroarylsulfonyl, such as phenylsulfonyl. In certain preferred embodiments of formula (II), R¹ is the payload or reactive moiety, and R³ is phenylsulfonyl. In other preferred embodiments, R¹ and R² are F, and R⁵ is the payload or reactive moiety.

In certain embodiments of formula (II), the payload or reactive moiety is a payload moiety. In certain embodiments, the payload moiety is a drug moiety, a detectable label, or a PEG moiety. In certain embodiments, the detectable label is a fluorophore, a radiolabel, or an imaging agent.

In certain embodiments of formula (II), the payload or reactive moiety is a reactive moiety. In certain embodiments, the reactive moiety is a hydroxyl, haloacyl, an alkene, an alkyl halide, an alkyne, an amine, an aryl azide, an aryl halide, an azide, a carbodiimide, a carboxyl, a diene, a dienophile, a glyoxal, an imidoester, an isocyanide, a maleimide, an N-hydroxysuccinimidyl (NHS) ester, a phosphine, a tetrazine, or a thiol. In certain embodiments, the reactive moiety is a haloacyl, an alkene, an alkyl halide, an alkyne, an amine, an aryl azide, an aryl halide, an azide, a carbodiimide, a carboxyl, a diene, a dienophile, a glyoxal, an imidoester, an isocyanide, a maleimide, an N-hydroxysuccinimidyl (NHS) ester, a phosphine, or a tetrazine.

In certain embodiments, the portion of formula (II) between A and B is a portion of a polycyclic structure selected from: (a) 2,4 methano-2,4-didehydroadamantane; (b) 1-(phenylsulfonyl)bicyclo[1.1.0]butane; (c) 2-methyl-bicyclo[1.1.0]butane; (d) tricyclo[4.1.0.0]heptane; or (e) 2,2-difluorobicyclo[1.1.0]butane. In certain preferred embodiments, the compound is:

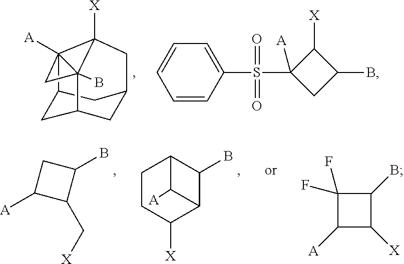

wherein X is the reactive or payload moiety.

The methods disclosed herein are applicable to modify or functionalize any type of peptide or protein that contains a disulfide bridge. Any form of payload (fluorophore, drug, PEG, etc.), can be attached to a linker as disclosed herein and readily conjugated to a biomolecule. An example of the utility of our method is making antibody-drug conjugates, which combine the specificity of monoclonal antibodies with the potency of cytotoxic drugs and have become an emerging field in the fight against cancer. Drugs conjugated to antibodies can be selectively delivered to harmful cells to reduce their effects on normal or beneficial cells.

### Discussion

Nature utilizes post-translational modification of proteins as one of the main methods for creating diversity in structure and thus in function. Scientists utilize the same approach in order to probe biological systems, invent novel drugs and gain a deeper understanding of biomolecules. However, there are many limitations while working within biological systems. An ideal conjugation strategy requires site selectivity, both chemo- and regioselectivity, in mild physiological conditions. For proteins, amino acids are the most convenient site for modification. One such easily exploited amino acid is cysteine because of its rare occurrence in proteins but also its reactive thiol group. However, many cysteines are tied up in disulfide bridges, which prompts a need for techniques to modify disulfide bonds. Most disulfide bonds play a crucial role in protein structure so modifications of these moieties need to be approached with care. Existing techniques to modify disulfide bonds require a reduction step to create free cysteine residues, which is liable to cause protein unfolding, aggregation and disulfide scrambling; ultimately ruining the structure and function of the protein.

The methods of the present disclosure use small strained systems to allow disulfide modification to occur selectively with one step, thus avoiding complications associated with functionalizing free cysteines and maintaining the crosslinking that disulfide bridges sometimes provide.

The value of strained molecules in bioconjugation strategies was exemplified by Bertozzi and coworkers with the use of cyclooctyne and azide click chemistry (Bertozzi et al. JACS 2004, 126, 15046). Furthermore, Majerski and coworkers have shown that 2,4-methano-2,3-didehydroadamantane, a small propellane with a bicyclobutane core, can react nearly instantaneously with a disulfide bond under mild conditions through a radical mechanism (Majerski et al. JOC 1989, 54, 545). The present disclosure shows that this strained bicyclobutane core can be used to modify disulfides in biomolecules, causing them to undergo homolytic cleavage for easy insertion within a disulfide bond, as depicted in Scheme 1 below:

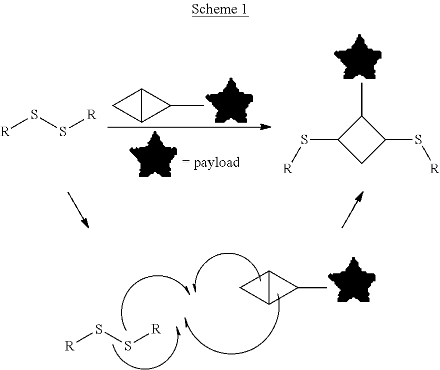

The strained central bond of the bicyclobutane moiety activates it for efficient selective addition into disulfide bonds. By addition of a payload, this invention provides a small bridged linker for general protein conjugation. This methodology is applicable to many forms of biomolecule modification, as long as disulfides are present, ranging from protein labeling for probing studies to antibody drug conjugates for novel therapeutics.

Antibodies are an exemplary biomolecule for this conjugation methodology, because of the prevalence of disulfides and their importance in forming and maintaining the characteristic Y shaped structure (see FIG. 3). Addition of a bicyclobutane molecule, such as a payload-linked bicyclo[1.1.0]butane-2-methanol, to an antibody causes a homolytic bond cleavage of the strained C—C bond of bicyclobutane, which readily combines with the weak disulfide bond as depicted in FIG. 3. This methodology provides an efficient one-step conjugation mechanism for linking any form of payload, such as a drug, to a biomolecule, such as an antibody.

In some embodiments, the conjugation reaction between the bicyclobutane compound and the protein will proceed without the need for additional reagents or catalysts. In other embodiments, a less reactive bicyclobutane molecule may be used that does not spontaneously react with the protein. In such embodiments, a photoinitiator may be used to initiate the reaction. For example, DMPA or IRGACURE 2959 may be added to the conjugation reaction mixture, and the reaction may be initiated by irradiation with light of the appropriate wavelength, e.g., 365 nm. In some embodiments, the bicyclobutane compound itself may be photosensitive or include a photosensitive moiety. For instance, the bicyclobutane compound may be functionalized with a photosensitive arylsulfonyl or heteroarylsulfonyl group.

Many bicyclobutane-containing compounds may now be envisioned that could be utilized for protein conjugation. Some exemplary compounds are depicted in Scheme 2: (a) 2,4-methano-2,4-didehydroadamantane, (b) 1-(phenylsulfonyl)bicyclo[1.1.0]butane, (c) bicyclo[1.1.0]butane-2-methanol, (d) tricyclo[4.1.0.0]heptan-3-ol (e) 2,2-difluorobicyclo[1.1.0]butane.

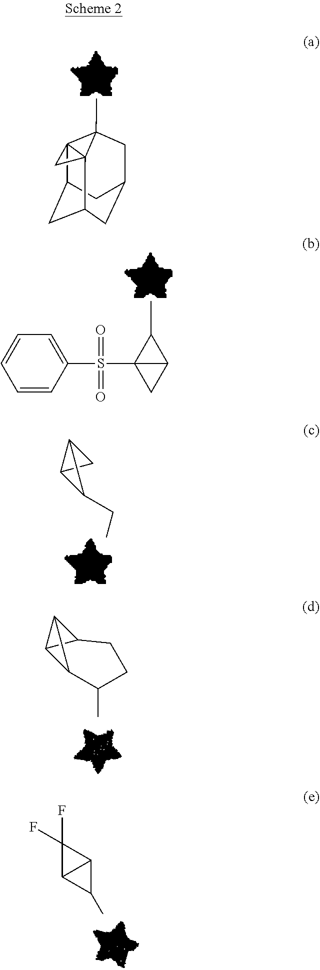

### Reactive Groups

Any reactive group known in the art may be used to couple the bicyclobutane moiety to the payload moiety. For instance, the reactive group may comprise an α-haloacyl, alkene, alkyl halide, alkyne, amine, aryl azide, aryl halides, azide, carbodiimide, carboxyl, diene, dienophile, glyoxal, imidoester, isocyanide, maleimide, N-hydroxysuccinimidyl (NHS) ester, phosphine, tetrazine, or thiol.

The reactive group may comprise an azide or alkyne, e.g., for coupling the payload moiety via the azide-alkyne Huisgen cycloaddition. Huisgen cycloaddition is the reaction of a dipolarophile with a 1,3-dipolar compound that leads to 5-membered (hetero)cycles. Examples of dipolarophiles are alkenes and alkynes and molecules that possess related heteroatom functional groups (such as carbonyls and nitriles). 1,3-Dipolar compounds contain one or more heteroatoms and can be described as having at least one mesomeric structure that represents a charged dipole. They include nitrile oxides, azides, and diazoalkanes. Metal catalyzed click chemistry is an extremely efficient variant of the Huisgen 1,3-dipolar cycloaddition reaction between alkyl, aryl, or sulfonyl azides, and terminal C—C triple bonds and C—N triple bonds, which is well-suited to the methods disclosed herein. The results of these reactions are 1,2-oxazoles, 1,2,3-triazoles or tetrazoles. For example, 1,2,3-triazoles are formed by a copper catalyzed Huisgen reaction between alkynes and alkyl/aryl azides. Metal-catalyzed Huisgen reactions proceed at ambient temperature, are not sensitive to solvents, i.e., nonpolar, polar, semipolar, and are highly tolerant of functional groups. Non-metal Huisgen reactions (also referred to as strain-promoted cycloaddition) involving use of a substituted cyclooctyne, which possesses ring strain and electron-withdrawing substituents such as fluorine, that together promote a [3+2] dipolar cycloaddition with azides are especially well-suited for use herein due to low toxicity of the reaction components as compared to the metal-catalyzed reactions. Examples include DIFO and DIMAC. Reaction of the alkynes and azides is very specific and essentially inert against the chemical environment of biological tissues.

The reactive group may comprise a diene or a dienophile, e.g., for coupling the payload moiety via the Diels-Alder reaction. The Diels-Alder reaction combines a diene (a molecule with two alternating double bonds) and a dienophile (an alkene) to make rings and bicyclic compounds. This could be a standard or inverse electron demand Diels-Alder reaction.

The reactive group may comprise an isocyanide or a tetrazine, e.g., for coupling the payload moiety via a 4+1 cycloaddition.

The reactive group may comprise a maleimide or a thiol, e.g., for coupling the payload moiety via a maleimide-thiol reaction (see, e.g., U.S. Patent Application Publication No. 2010/0036136, hereby incorporated by reference).

The reactive group may comprise a phosphine or an azide, e.g., for coupling the payload moiety via the Staudinger reaction. The classical Staudinger reaction is a chemical reaction in which the combination of an azide with a phosphine or phosphite produces an aza-ylide intermediate, which upon hydrolysis yields a phosphine oxide and an amine. A Staudinger reaction is a mild method of reducing an azide to an amine; and triphenylphosphine is commonly used as the reducing agent. In a Staudinger ligation, an electrophilic trap (usually a methyl ester) is appropriately placed on a triarylphosphine (usually in ortho to the phosphorus atom) and reacted with the azide, to yield an aza-ylide intermediate, which rearranges in aqueous media to produce a compound with amide group and a phosphine oxide function. The Staudinger ligation is so named because it ligates (attaches/covalently links) the two starting molecules together, whereas in the classical Staudinger reaction, the two products are not covalently linked after hydrolysis.

Reactive groups can be joined to the bicyclobutane moiety through varying lengths of spacer arms or bridges, which may be alkyl chains, PEG chains, amino-acid chains, or any other suitable spacers. For example, a linker may comprise a polyethylene glycol (PEG) chain, e.g., which serves as a spacer between the functional group and the agent. A linker may comprise, for example, diethylene glycol, triethylene glycol, tetraethylene glycol, pentaethylene glycol, or hexaethylene glycol. A linker may comprise, for example a first functional group (e.g., R¹), a polyethylene glycol spacer (e.g., —[OCH₂CH₂]ₙO—, wherein n is an integer from 2 to 100, such as 2 to 50, or 2 to 20), and a second functional group (e.g., R²). For example, the linker may be a molecule of formula R¹[OCH₂CH₂]ₙOR² or R²[OCH₂CH₂]ₙOR¹. R¹ and R² may each independently be selected from a moiety comprising an α-haloacyl, alkene, alkyl halide, alkyne, amine, aryl azide, aryl halides, azide, carbodiimide, carboxyl, diene, dienophile, glyoxal, imidoester, isocyanide, maleimide, N-hydroxysuccinimidyl (NHS) ester, phosphine, tetrazine, or thiol. R¹ may be thiol and R² may be carboxyl, or R² may be thiol and R¹ may be carboxyl. R¹ may be thiol and R² may be amine, or R² may be thiol and R¹ may be amine. R¹ may be carboxyl and R² may be amine, or R² may be carboxyl and R¹ may be amine.

Reactive groups and functional groups suitable for reacting with primary amines include imidoesters and N-hydroxysuccinimidyl (NHS) esters. Examples of imidoester functional groups include dimethyladipimidate, dimethylpimelimidate, and dimethylsuberimidate. Examples of NHS-ester functional groups include disuccinimidyl glutamate, disuccinimidyl suberate, and bis(sulfosuccinimidyl) suberate. Accessible amine groups present on the N-termini of peptides, polypeptides, and proteins react with NETS-esters to form amides. NETS-ester cross-linking reactions can be conducted in phosphate, bicarbonate/carbonate, HEPES and borate buffers. Other buffers can be used if they do not contain primary amines. The reaction of NHS-esters with primary amines may be conducted at a pH of between about 7 and about 9 and a temperature between about 4° C. and 30° C. for about 30 minutes to about 2 hours. The concentration of NHS-ester functional group may vary from about 0.1 to about 10 mM. NETS-esters are either hydrophilic or hydrophobic. Hydrophilic NETS-esters are reacted in aqueous solutions although DMSO may be included to achieve greater solubility. Hydrophobic NHS-esters are dissolved in a water miscible organic solvent and then added to the aqueous reaction mixture.

Sulfhydryl-reactive functional groups and reactive groups include maleimides, alkyl halides, aryl halides, and a-haloacyls which react with sulfhydryls to form thiol ether bonds and pyridyl disulfides which react with sulfhydryls to produce mixed disulfides. Sulfhydryl groups on peptides, polypeptides, and proteins can be generated by techniques known to those with skill in the art, e.g., by reduction of disulfide bonds or addition by reaction with primary amines using 2-iminothiolane. Examples of maleimide functional groups include succinimidyl 4-{N-maleimido-methyl)cyclohexane-1-carboxylate and m-maleimidobenzoyl-N-hydroxysuccinimide ester. Examples of haloacetal functional groups include N-succinimidyl (4-iodoacetal) aminobenzoate and sulfosuccinimidyl (4-iodoacetal) aminobenzoate. Examples of pyridyl disulfide functional groups include 1,4-di-[3′-2′-pyridyldithio(propionamido)butane] and N-succinimidyl-3-(2-pyridyldithio)-propionate.

The reactive group may be a carboxyl groups for binding to primary amines or hydrazides by using carbodiimides, which result in formation of amide or hydrazone bonds. Examples of carbodiimide functional groups and reactive groups include 1-ethyl-3-(3-dimethylaminopropyl)-carbodiimide hydrochloride and N,N′-dicyclohexylcarbodiimide. Arylazide becomes reactive when exposed to ultraviolet radiation to form aryl nitrene. Examples of arylazide functional groups include azidobenzoyl hydrazide and N-5-azido-2 nitrobenzoyloxysuccinimide. Glyoxal functional groups target the guanidyl portion of arginine. An example of a glyoxal functional group is p-azidophenyl glyoxal monohydrate.

### Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

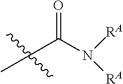

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

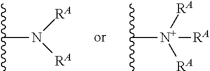

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

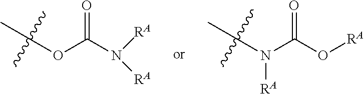

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

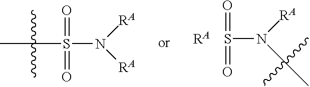

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or —SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

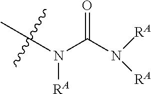

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3rd Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

### EXAMPLES

The invention now being generally described, it will be more readily understood by reference to the following examples which are included merely for purposes of illustration of certain aspects and embodiments of the present invention, and are not intended to limit the invention.

### Example 1: Synthesis of Functionalized 2,4-methano-2,4-didehydroadamantane

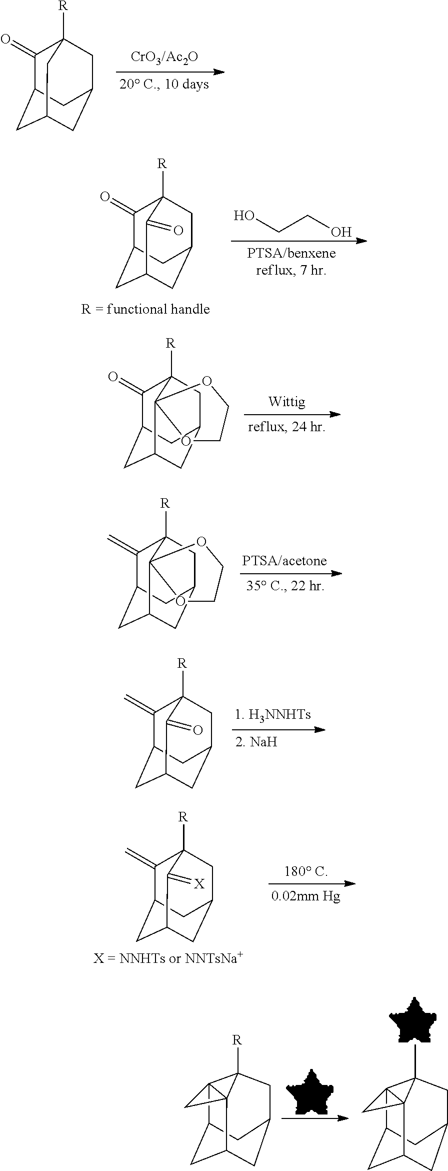

Starting with a functionalized 2-adamantanone, oxidation with chromium oxide in acetic anhydride provides adamantane-2,4-dione (Gilbert E., Syn. Comm. 1985, 15, 53). The procedure of Chu and coworkers may be followed to provide the corresponding functionalized 4-methyleneadamantan-2-one (Chu et al. Tetrahedron 2004, 60, 9493). From there, the procedure of Majerski et al. may be followed to provide the corresponding functionalized 2,4-methano-2,4-didehydroadamantane (Majerski et al. JACS 1983, 105, 7389). The functional handle may be a payload moiety, or may be an appropriate reactive moiety for coupling a payload moiety after preparation of the functionalized compound. A number of reactive moieties are disclosed herein, and may be selected according to the type of payload moiety that is to be attached. The reactive moiety may then be conjugated to the payload moiety by any suitable method, for example as disclosed herein.

### Example 2: Synthesis of 1-(phenylsulfonyl)bicyclo[1.1.0]butane

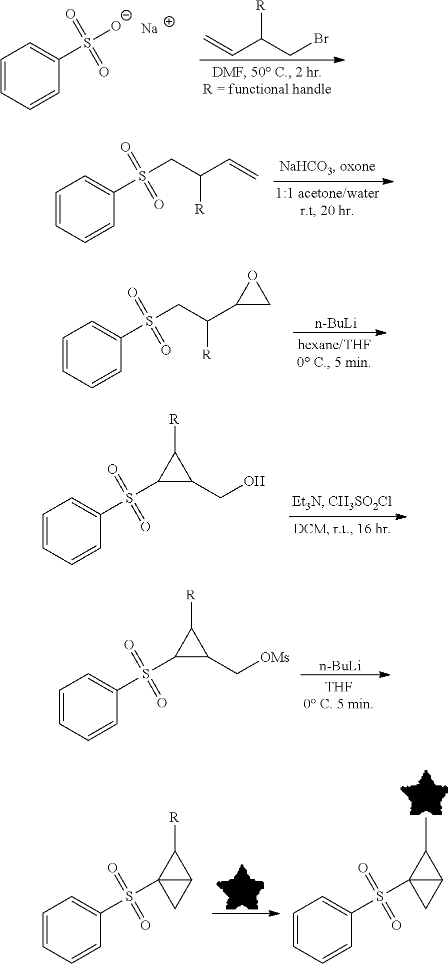

1-(Phenylsulfonyl)bicyclo[1.1.0]butane was prepared by an analogous method to that of Baran et al. (Gianatassio R.; Baran P. S. et al. Science, 2016, 351, 241). ¹H-NMR matched literature. ¹H NMR (400 MHz, Chloroform-d) δ 8.00-7.79 (m, 2H), 7.68-7.47 (m, 3H), 2.57 (d, J=3.3 Hz, 1H), 2.54-2.51 (m, 2H), 1.39 (dt, J=2.7, 0.9 Hz, 2H).

2-Functionalized 1-(phenylsulfonyl)bicyclo[1.1.0]butane may be prepared analogously. The functional handle may be a payload moiety, or may be an appropriate reactive moiety for coupling a payload moiety after preparation of the functionalized compound. A number of reactive moieties are disclosed herein, and may be selected according to the type of payload moiety that is to be attached. The reactive moiety may then be conjugated to the payload moiety by any suitable method, for example as disclosed herein.

### Example 3: Synthesis of Bicyclo[1.1.0]butane-2-methanol

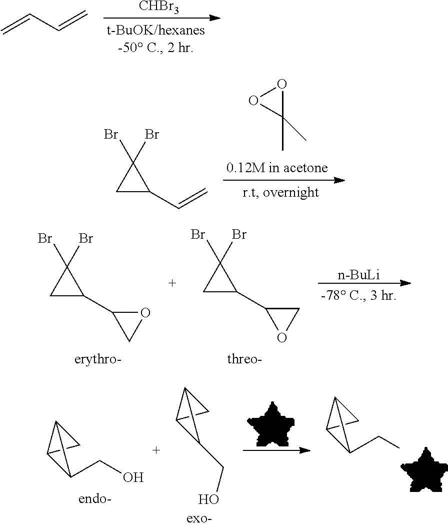

Bicyclo[1.1.0]butane-2-methanol was prepared as described by Bentley et al. (Bentley et al. J. Org. Chem. 2006, 71, 1018). ¹H-NMR matched literature. Endo: ¹H NMR (400 MHz, Chloroform-d) δ 3.37 (dd, J=7.4, 5.9 Hz, 2H), 2.69 (dq, J=7.3, 3.6 Hz, 1H), 1.85 (dd, J=3.3, 1.9 Hz, 1H), 1.44 (d, J=1.6 Hz, 1H). Exo: ¹H NMR (400 MHz, Chloroform-d) δ 3.56 (t, J=5.8 Hz, 2H), 1.51 (td, J=3.1, 0.8 Hz, 1H), 1.45 (dt, J=3.0, 1.0 Hz, 2H), 0.62-0.58 (m, 1H). The prepared compound may be conjugated to a payload moiety by any suitable method.

### Example 4: Synthesis of Tricyclo[4.1.0.0]heptan-3-ol

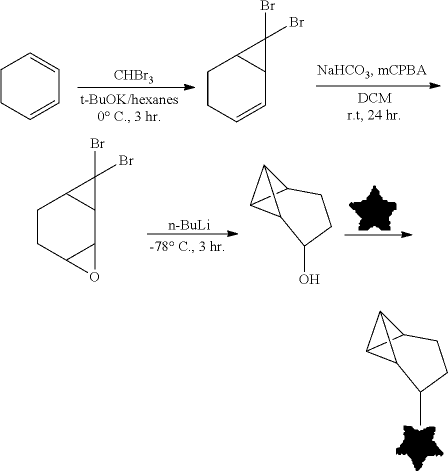

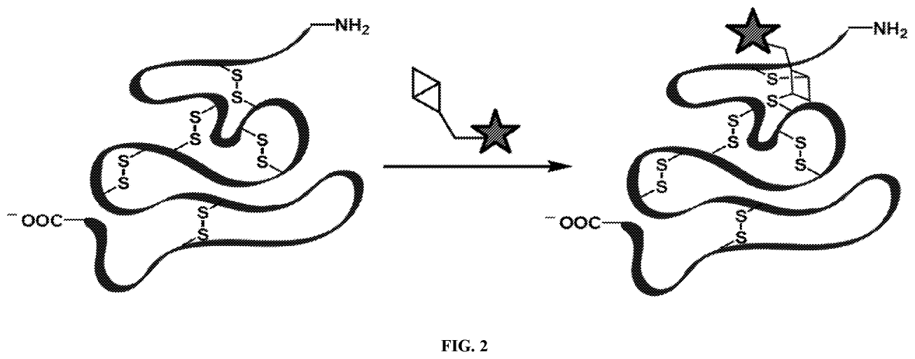

Tricyclo[4.1.0.0]heptan-3-ol (FIG. 2d) is prepared in an analogous method to bicyclo[1.1.0]butane-2-methanol, starting with 1,3-cyclohexadiene. It may then be conjugated to a payload moiety by any suitable method.

### Example 5: Synthesis of 2,2-diflurorbicyclobutane

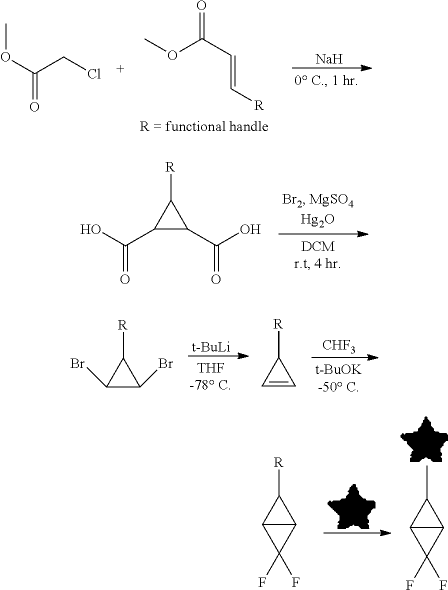

The addition of sodium hydride to a solution of methyl 2-chloropropionate and a functionalized ethyl methacrylate at 0° C. provides functionalized 1,2-cyclopropanedicarboxylic acid after saponification (McCoy L. JACS 1958, 80, 6569). A modified Hunsdiecker reaction then provides functionalized 1,2-dibromocyclopropane (Wiberg et al. JACS 1991, 113, 7969). To achieve the cyclopropene structure, tert-butyllithium is added at −78° C. The addition of chloroform via a Doering-Hoffmann method provides functionalized 2,2-difluorobicyclo[1.1.0]butane (Wiberg K. B. and Bonneville G. Tet. Lett. 1982, 23, 5385). The functional handle may be a payload moiety, or may be an appropriate reactive moiety for coupling a payload moiety after preparation of the functionalized compound. A number of reactive moieties are disclosed herein, and may be selected according to the type of payload moiety that is to be attached. The reactive moiety may then be conjugated to the payload moiety by any suitable method, for example as disclosed herein.

### Example 6: Synthesis of 1-(phenylsulfonyl)bicyclo[1.1.0]butane

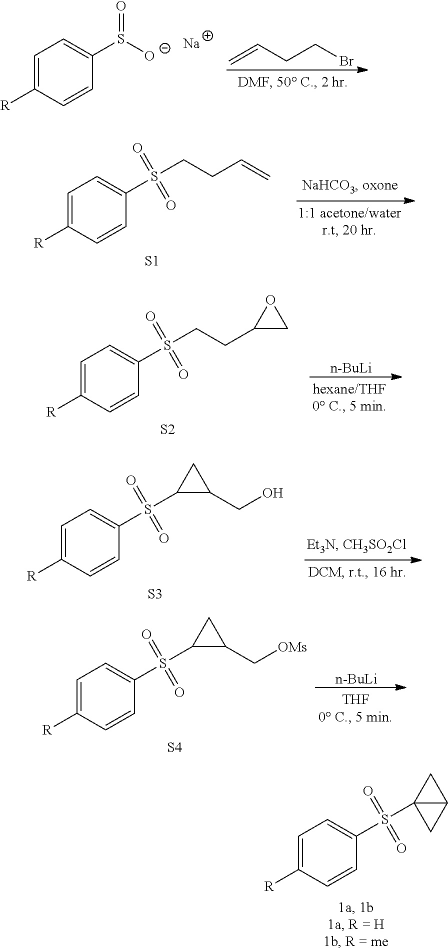

But-3-en-1-ylsulfonylbenzene (S1a) Sodium benzene sulfinate (50 mg, 0.28 mmol, 1 eq) was dissolved in DMF (dimethylformamide) (0.40 mL, anhydrous) and 4-bromobut-1-ene (0.040 mL, 0.33 mmol, 1.2 eq) was added dropwise. The reaction was heated to 50° C. After 2 h, the reaction was diluted with ethyl acetate (3 mL), washed with brine (1×5 mL), dried over MgSO₄, filtered and evaporated to dryness. The crude product was purified via silica gel chromatography with hexane:ethyl acetate (15:1→10:1→5:1) to afford pure but-3-en-1-ylsulfonylbenzene (13.8 mg, 0.061 mmol, 22% yield). ¹H-NMR matched literature values. ¹H NMR (500 MHz, Chloroform-d) δ 7.92 (dd, J=8.4, 1.3 Hz, 2H), 7.69-7.65 (m, 1H), 7.60-7.56 (m, 2H), 5.73 (ddt, J=16.8, 10.2, 6.5 Hz, 1H), 5.09-5.02 (m, 2H), 3.21-3.13 (m, 2H), 2.50-2.42 (m, 2H).

(2-phenylsulfonylethyl)oxirane (S2a) But-3-en-1-ylsulfonylbenzene (S1a) (100 mg, 6.51 mmol, 1.0 eq) was dissolved in H₂O (1.7 mL) and acetone (1.7 mL) along with NaHCO₃ (214 mg, 2.55 mmol, 5 eq). Oxone (407 mg, 1.32 mmol, 2.6 eq) was added portion wise over 4 h. After 6 h, reaction was evaporated to dryness, diluted with ethyl acetate (5 mL), washed with brine (2×5 mL), dried over MgSO₄, filtered and evaporated to afford pure (2-phenylsulfonylethyl)oxirane. ¹H-NMR matched literature values.

(2-(phenylsulfonyl)cyclopropyl)methanol (S3a) (2-phenyl sulfonylethyl)oxirane S2 (432 mg, 2.04 mmol, 1 eq) was dissolved in THF (tetrahydrofuran) (10 mL, anhydrous). Reaction was cooled to 0° C. n-BuLi (0.83 M in hexanes, 2.45 mL, 2.04 mmol, 1 eq) was added dropwise over 30 min. The solution changed from clear to pale yellow upon addition of n-BuLi. After 1 h, reaction was quenched with a saturated solution of NH₄Cl, extracted with ethyl acetate (3×10 mL), dried over MgSO₄, filtered and evaporated to afford (2-(phenylsulfonyl)cyclopropyl)methanol (278 mg, 1.31 mmol, 64%). ¹H-NMR matched literature values.^{i 1}H NMR (400 MHz, CDCl₃) δ 7.91 (d, J=20.9 Hz, 2H), 7.68-7.62 (m, 1H), 7.60-7.53 (m, 2H), 3.71 (dt, J=11.2, 5.5 Hz, 1H), 3.55 (dt, J=11.3, 5.6 Hz, 1H), 2.46 (dt, J=8.3, 4.7 Hz, 1H), 2.10-2.05 (m, 1H), 1.51 (dt, J=10.4, 4.4 Hz, 2H), 1.32 (t, J=5.7 Hz, 1H), 1.10 (ddd, J=8.3, 6.4, 5.4 Hz, 1H).

(2-(phenylsulfonyl)cyclopropyl)methyl methanesulfonate (S4a) (2-(phenylsulfonyl)cyclopropyl)methanol S3 (278 mg, 1.31 mmol, 1 eq) was dissolved in dichloromethane (0.35 mL, anhydrous). NEt₃ (37 μL, 1.1 eq) and methane sulfonyl chloride (20.08 μL, 1.1 eq) were added sequentially at 0° C. The reaction mixture was warmed to room temperature and stirred for 1 h, at which point the reaction was quenched with dichloromethane and washed with brine (3×5 mL), dried over MgSO₄, filtered and evaporated to dryness to give a colorless liquid. The crude product was dry loaded onto silica with dichloromethane and then purified by silica gel chromatography in 3:2 hexane:ethyl acetate to afford 1-(phenylsulfonyl)bicyclo[1.1.0]butane (274 mg, 0.94 mmol, 72%). ¹H-NMR matched literature values. ¹H NMR (400 MHz, Chloroform-d): δ 7.93-7.89 (m, 2H), 7.70-7.64 (m, 1H), 7.62-7.55 (m, 2H), 4.30 (dd, J=11.3, 5.9 Hz, 1H), 3.98 (dd, J=11.3, 7.7 Hz, 1H), 2.95 (s, 3H), 2.58 (ddd, J=8.6, 5.3, 4.3 Hz, 1H), 2.25-2.11 (m, 1H), 1.66 (ddd, J=9.5, 5.9, 5.3 Hz, 1H), 1.17 (dt, J=8.5, 6.1 Hz, 1H).

1-(phenylsulfonyl)bicyclo[1.1.0]butane (1a) (2-(phenylsulfonyl)cyclopropyl)methyl methanesulfonate S4 (343 mg, 1.18 mmol, 1 eq) was dissolved in THF (5.70 mL, anhydrous). Reaction was cooled to 0° C. n-BuLi (0.83 M in hexanes, 1.42 mL, 1.18 mmol, 1 eq) was added dropwise. The solution went from clear to dark brown upon addition of n-BuLi. After 5 min, reaction was quenched with a saturated solution of NH₄Cl, extracted with dichloromethane (3×10 mL), dried over MgSO₄, filtered and evaporated to dryness. The crude product was purified via silica gel chromatography with hexane:ethyl acetate (20:1) to afford pure 1-(phenylsulfonyl)bicyclo[1.1.0]butane (70 mg, 0.36 mmol, 30%). ¹H-NMR matched literature. ¹H NMR (400 MHz, Chloroform-d) δ 8.00-7.79 (m, 2H), 7.68-7.47 (m, 3H), 2.57 (d, J=3.3 Hz, 1H), 2.54-2.51 (m, 2H), 1.39 (dt, J=2.7, 0.9 Hz, 2H).

The synthesis of 1-tosylbicyclo[1.1.0]butane (1b) was performed following the same above procedures but with sodium p-toluenesulfinate salt as the starting material.

1-(but-3-en-1-ylsulfonyl)-4-methylbenzene (Sib) ¹H-NMR matched literature. ¹H NMR (400 MHz, Chloroform-d) δ 7.59 (d, J=8.2 Hz, 2H), 7.37-7.29 (m, 2H), 5.73 (ddt, J=17.0, 10.3, 6.7 Hz, 1H), 5.11 (q, J=1.6 Hz, 1H), 5.09-5.04 (m, 1H), 4.07 (dt, J=9.9, 6.8 Hz, 1H), 3.64 (dt, J=9.9, 6.7 Hz, 1H), 2.42 (s, 3H), 2.38 (qt, J=6.7, 1.4 Hz, 2H).

2-(2-tosylethyl)oxirane (S2b)¹H-NMR matched literature. ¹H NMR (400 MHz, Chloroform-d) δ 7.79 (d, J=8.3 Hz, 2H), 7.40-7.33 (m, 2H), 3.20 (ddd, J=8.5, 6.6, 1.4 Hz, 2H), 2.99 (dtd, J=6.6, 4.1, 2.6 Hz, 1H), 2.77 (dd, J=4.8, 3.9 Hz, 1H), 2.49 (dd, J=4.8, 2.6 Hz, 1H), 2.45 (s, 4H), 2.16-2.09 (m, 1H), 1.88-1.76 (m, 1H).

(2-tosylcyclopropyl)methanol (S3b)¹H-NMR matched literature. ¹H NMR (300 MHz, Chloroform-d) δ 7.83 (d, J=8.3 Hz, 2H), 7.39 (d, J=8.1 Hz, 2H), 3.74 (dt, J=11.3, 5.6 Hz, 1H), 3.57 (dt, J=11.4, 5.7 Hz, 1H), 2.49 (s, 3H), 2.14-2.01 (m, 1H), 1.54 (dt, J=9.4, 5.2 Hz, 1H), 1.38 (t, J=5.9 Hz, 1H), 1.11 (ddd, J=8.4, 6.5, 5.5 Hz, 1H).

(2-tosylcyclopropyl)methyl methanesulfonate (S4b)¹H-NMR matched literature. ¹H NMR (400 MHz, Chloroform-d) δ 7.78 (d, J=8.3 Hz, 2H), 7.40-7.32 (m, 2H), 4.27 (dd, J=11.3, 6.0 Hz, 1H), 4.01 (dd, J=11.2, 7.5 Hz, 1H), 2.96 (s, 4H), 2.56 (ddd, J=8.5, 5.3, 4.3 Hz, 1H), 2.46 (s, 4H), 2.22-2.10 (m, 1H), 1.63 (ddd, J=9.5, 5.8, 5.2 Hz, 1H), 1.14 (dt, J=8.5, 6.0 Hz, 1H).

1-tosylbicyclo[1.1.0]butane (1b)¹H-NMR matched literature. ¹H NMR (400 MHz, Chloroform-d) δ 7.82 (d, J=8.4 Hz, 2H), 7.35 (dt, J=8.0, 0.7 Hz, 2H), 2.60-2.47 (m, 3H), 2.45 (s, 4H), 1.36 (dt, J=2.6, 0.8 Hz, 2H).

### Example 7: Synthesis of Bicyclo[1.1.-]butane-2-methanol

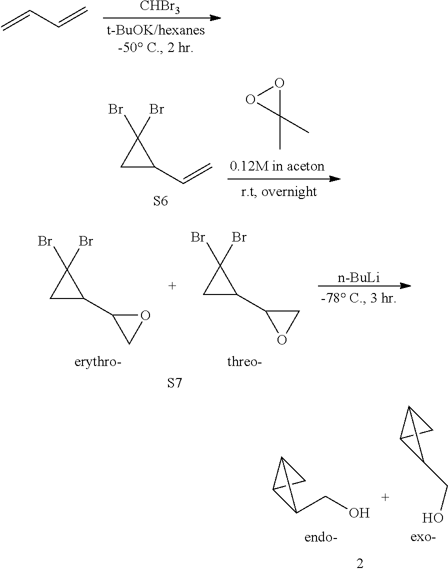

1,1-dibromo-2-vinylcyclopropane (S6) A two-neck round bottom flask was attached to a Dewar condenser filled with a mixture of dry ice and acetone. t-BuOK (405 mg, 3.70 mmol. 0.8 eq) was added and the reaction mixture was cooled to −50° C. as 1,3 butadiene (20 wt % in toluene, 1.55 mL, 4.62 mmol, 1 eq) was added slowly. Bromoform (0.310 mL, 4.62 mmol, 1 eq) in a solution of hexane (0.660 mL, anhydrous) was added dropwise over an hour. After 1 hour at −50° C., the reaction was warmed to room temperature. After an additional 1 h, reaction was quenched with H₂O, extracted with hexanes (3×10 mL), dried over MgSO₄, filtered and evaporated to dryness. The crude product was purified via silica gel chromatography with hexanes: ethyl acetate (20:1) to afford pure 1,1-dibromo-2-vinylcyclopropane (70 mg, 0.36 mmol, 30%). ¹H′NMR matched literature. ¹H NMR (400 MHz, Chloroform-d) δ 5.57 (ddd, J=17.0, 10.1, 8.1 Hz, 1H), 5.37 (dd, J=1.5, 0.7 Hz, 1H), 5.34-5.28 (m, 1H), 2.34-2.25 (m, 1H), 1.97 (dd, J=10.2, 7.4 Hz, 1H), 1.58 (t, J=7.6 Hz, 1H)

Erythro-(erythro-S7) and threo-(threo-S7) 2′,2′-dibromocycloprop-1′-yloxirane DMDO (0.12M in acetone, 11.70 mL, 1.40 mmol, 1 eq) was added slowly over 1 hr to 1,1-dibromo-2-vinylcyclopropane S6 (311 mg, 1.40 mmol, 1 eq) at 0° C. Reaction was warmed to room temperature and left to stir overnight. Reaction was evaporated to dryness. The crude product was purified via silica gel chromatography with pentane:diethyl ether (25:1-15:1→9:1) to afford pure erythro-2′,2′-dibromocycloprop-1′-yloxirane (86 mg, 0.35 mmol, 26%)¹H NMR (400 MHz, Chloroform-d) δ 2.94 (ddd, J=6.6, 4.0, 2.6 Hz, 1H), 2.86 (dd, J=4.8, 4.0 Hz, 1H), 2.69 (dd, J=4.9, 2.6 Hz, 1H), 1.84 (dd, J=10.0, 7.1 Hz, 1H), 1.63-1.58 (m, 1H), 1.54 (q, J=7.5 Hz, 1H) and threo-2′,2′-dibromocycloprop-1′-yloxirane (61 mg, 0.26 mmol, 18%). ¹H′NMR matched literature. ¹H NMR (400 MHz, Chloroform-d) δ 3.09 (td, J=3.8, 2.6 Hz, 1H), 2.89 (dd, J=5.1, 3.9 Hz, 1H), 2.63 (dd, J=5.1, 2.6 Hz, 1H), 1.85 (ddd, J=10.3, 7.4, 3.7 Hz, 1H), 1.75 (dd, J=10.3, 7.2 Hz, 1H), 1.65-1.60 (m, 1H).

Endo-Bicyclo[1.1.-]butane-2-methanol (endo-2) n-BuLi (1.6 M in hexanes, 0.3 mL, 0.42 mmol, 2 eq) was added over 30 minutes to a solution of threo-2′,2′-dibromocycloprop-1′-yloxirane (threo-S7) (50 mg, 0.21 mmol, 1 eq) in diethyl ether (3.7 mL, anhydrous) at −78° C. After 1 h at −78° C., reaction was warmed to room temperature. After an additional 1 h, reaction was quenched with H₂O, and extracted with diethyl ether (3×5 mL), washed with brine (1×5 mL), dried over MgSO₄, filtered and evaporated to dryness to afford crude endo-Bicyclo[1.1.-]butane-2-methanol (20 mg, 0.23 mmol, 57%). ¹H-NMR matched literature. ¹H NMR (400 MHz, Chloroform-d) δ 3.37 (dd, J=7.4, 5.9 Hz, 2H), 2.69 (dq, J=7.3, 3.6 Hz, 1H), 1.85 (dd, J=3.3, 1.9 Hz, 1H), 1.44 (d, J=1.6 Hz, 1H).

Exo-Bicyclo[1.1.-]butane-2-methanol (exo-2) Similar procedure was utilized for erythro-S7 as threo-S7 to afford Exo-Bicyclo[1.1.-]butane-2-methanol. ¹H-NMR matched literature. ¹H NMR (400 MHz, Chloroform-d) δ 3.56 (t, J=5.8 Hz, 2H), 1.51 (td, J=3.1, 0.8 Hz, 1H), 1.45 (dt, J=3.0, 1.0 Hz, 2H), 0.62-0.58 (m, 1H).

### Example 8: Stability of Bicyclobutanes

Previous bicyclobutane molecules (1a, 1b) were determined by Baran and coworkers to be bench stable so no further studies were conducted on their stability. 1a and 1b were found to not be completely soluble in polar solvents (acetonitrile, methanol).

To determine the stability and selectivity of bicyclobutane molecules endo-3, exo-3 were solubilized in diethyl ether and tested with a variety of functional groups (diphenyl disulfide, benzyl amine, diphenyl acetic acid, and 1-butanethiol) then analyzed on a GC-MS to confirm the continued presence of starting materials. A reaction with 3 was apparent only in the case with diphenyl acetic acid and 1-butane thiol, confirming the relative stability of the molecule.

Solubility in polar solvents is important if this linker is to be used as a bioconjugation strategy, thus endo-3, exo-3 were confirmed to be soluble in a 9:1 methanol:water mixture. ¹H-NMR studies of these molecules in solvent left on the bench showed no change over 2 days suggesting stability of these molecules in polar solvents.

### Example 9: Reactivity with Disulfides

Three solutions of 1a in CDCl₃ were prepared. Solution A: control. Solution B: diphenyl disulfide (5 eq). Solution C: n-hexyl disulfide (5 eq).

Each solution was irradiated with 365 nm light in a photobox (>5000 μW/cm² at 38 cm). After 1 h of irradiation, solution showed loss of starting material and LCMS traces showed the presence of a double addition (3) and mono addition (4). However no reaction was apparent in solution B or C.

A similar method was performed as above with the addition of 10 wt % DMPA (2,2-diemethoxy-2-pheynlacetophenone, a photoinitiator) which showed similar results with a ratio of 40%: 60% of 3:4.

Similar studies with 1 b showed similar reactivity as above (FIG. 2b).

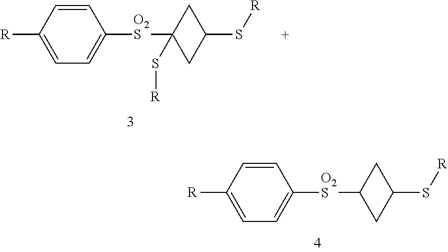

Three solutions of endo-3, exo-3 in 9:1 CD₃OD-D₂O were prepared. Solution A: control. Solution B: L-cysteine HCl (5 eq). Solution C: L-cysteine dimethyl ester 2HCl (5 eq). Solution D: Dl-alanine (5 eq). Each was irradiated with 365 nm light in a photobox (>5000 μW/cm² at 38 cm). After 24 h of irradiation, solution C showed loss of starting material but unidentifiable products.

### Example 10: Conjugation of a Diphenyldisulfide

1-(phenylsulfonyl)bicyclo[1.1.0]butane was prepared according to the methods disclosed herein and reacted with diphenyldisulfide both in the absence and presence of the photosensitizer DMPA. As seen in table 1 below, the photosensitizer increased the yield of the desired transformation. 1-(4-methyl phenylsulfonyl)bicyclo[1.1.0]butane also provided the desired product.

TABLE 1

| 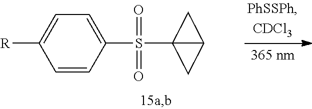 | | | |
|---|---|---|---|
| 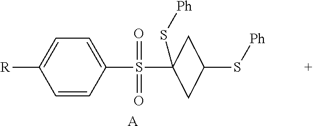 | | | |
| 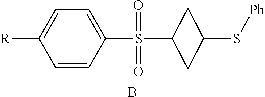 | | | |
| Entry | R | Photoinitiator | Product Ratio (A:B) |
| 1 | H | N/A | 20%:80% |
| 2 | H | DMPA | 40%:60% |
| 3 | Me | N/A | 30%:70% |

### Example 11: Conjugation of a Protein

A 1-(phenylsulfonyl)bicyclo[1.1.0]butane is prepared according to the methods disclosed herein with a drug moiety coupled at the 2-position. Separately, an antibody is prepared in aqueous solution. Preferably, the solution is buffered at a pH of between 4-8. The 2-functionalized 1-(phenylsulfonyl)bicyclo[1.1.0]butane is added to the antibody solution. Preferably, the reaction is conducted at a temperature from 0-60° C. Optionally, a photoinitiator is added and the solution is irradiated, for instance by light with a wavelength of 365 nm.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound comprising a bicyclobutane moiety coupled to a payload or reactive moiety, wherein the bicyclobutane moiety is represented by formula I: 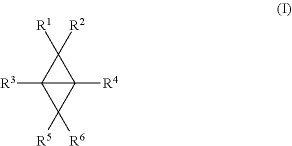
wherein:
R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, heteroarylsulfonyl, chromophore, fluorophore, or the payload or reactive moiety; and
further wherein any of R¹, R², R³, R⁴, R⁵, and R⁶, taken together with the carbon atoms that separate them, optionally complete one or more rings.

2. (canceled)

3. The compound of claim 1, wherein the bicyclobutane moiety is represented by formula I: 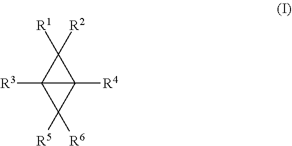
wherein:
R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, heteroarylsulfonyl, or the payload or reactive moiety; and
further wherein any of R¹, R², R³, R⁴, R⁵, and R⁶, taken together with the carbon atoms that separate them, optionally complete one or more rings.

4. The compound of claim 1, wherein the bicyclobutane moiety is represented by formula I: 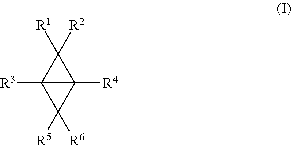
wherein:
R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, heteroarylsulfonyl, or the payload or reactive moiety; and
further wherein any of R¹, R², R³, R⁴, R⁵, and R⁶, taken together with the carbon atoms that separate them, complete one or more rings.

5. The compound of claim 1, wherein R¹, R², R³, R⁴, R⁵, and R⁶ are independently selected from H, alkyl, or the payload or reactive moiety; and further wherein any of R¹, R², R³, R⁴, R⁵, and R⁶, together with the carbon atoms that separate them, complete one or more rings.

6. The compound of claim 1, wherein at least one of R¹, R², R³, R⁴, R⁵, and R⁶ is the payload or reactive moiety.

7. The compound of claim 1, wherein at least one of R², R³, R⁴, R⁵, and R⁶ is coupled to the payload or reactive moiety.

8. The compound of claim 1, wherein at least one of R¹, R², R³, R⁴, R⁵, and R⁶ is arylsulfonyl or heteroarylsulfonyl, such as phenylsulfonyl.

9. The compound of claim 8, wherein R¹ is the payload or reactive moiety, and R³ is phenylsulfonyl.

10. The compound of claim 1, wherein at least one of R¹, R², R³, R⁴, R⁵ and R⁶ is halo.

11. The compound of claim 10, wherein R¹ and R² are F, and R⁵ is the payload or reactive moiety.

12. The compound of claim 1, wherein the payload or reactive moiety is a payload moiety.

13. The compound of claim 12, wherein the payload moiety is a drug moiety, a detectable label, or a PEG moiety.

14. The compound of claim 13, wherein the detectable label is a fluorophore, a radiolabel, or an imaging agent.

15. The compound of claim 1, wherein the payload or reactive moiety is a reactive moiety.

16. The compound of claim 15, wherein the reactive moiety is a hydroxyl, haloacyl, an alkene, an alkyl halide, an alkyne, an amine, an aryl azide, an aryl halide, an azide, a carbodiimide, a carboxyl, a diene, a dienophile, a glyoxal, an imidoester, an isocyanide, a maleimide, an N-hydroxysuccinimidyl (NHS) ester, a phosphine, a tetrazine, or a thiol.

17. The compound of claim 15, wherein the reactive moiety is a haloacyl, an alkene, an alkyl halide, an alkyne, an amine, an aryl azide, an aryl halide, an azide, a carbodiimide, a carboxyl, a diene, a dienophile, a glyoxal, an imidoester, an isocyanide, a maleimide, an N-hydroxysuccinimidyl (NHS) ester, a phosphine, or a tetrazine.

18. The compound of claim 1, wherein the bicyclobutane moiety is a portion of a polycyclic structure selected from:
(a) 2,4 methano-2,4-didehydroadamantane;
(b) 1-(phenylsulfonyl)bicyclo[1.1.0]butane;
(c) 2-methyl-bicyclo[1.1.0]butane;
(d) tricyclo[4.1.0.0]heptane; or
(e) 2,2-difluorobicyclo[1.1.0]butane.

19. The compound of claim 1, wherein the compound is: 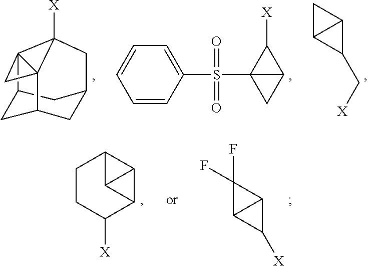
wherein X is the reactive or payload moiety.

20. A method for preparing a conjugated biomolecule, comprising:
providing a biomolecule comprising a disulfide bridge; and
reacting the disulfide bridge with a compound of claim 1, thereby producing the conjugated biomolecule.

21-23. (canceled)

24. A modified polypeptide comprising a moiety according to formula II: 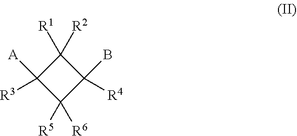
wherein:
A and B are sulfur atoms of cysteine residues of the polypeptide, wherein the sulfur atoms are capable of forming a disulfide bond in an unmodified form of the polypeptide;
R¹, R², R³, R⁴, R⁵, and R⁶ are independently selected from H, alkyl, alkenyl, alkynyl, cycloalkyl, heterocycloalkyl, aryl, heteroaryl, halo, cyano, hydroxy, alkylsulfonyl, arylsulfonyl, heteroarylsulfonyl, a chromophore, a fluorophore, or the payload or reactive moiety; and
further wherein any of R², R³, R⁴, R⁵, and R⁶, together with the carbon atoms that separate them, optionally complete one or more rings.

25-42. (canceled)
